(19) United States
(12) Patent Application Publication
(10) Pub. No.: US 2014/0018772 A1
(43) Pub. Date: Jan. 16, 2014

(54) SELF-CENTERING CATHETER WITH ANTI-OCCLUSION FEATURES

(71) Applicant: Stephen R. Ash, Lafayette, IN (US)

(72) Inventor: Stephen R. Ash, Lafayette, IN (US)

(73) Assignee: MERIT MEDICAL SYSTEMS, INC., South Jordan, UT (US)

(21) Appl. No.: 13/550,248

(22) Filed: Jul. 16, 2012

(51) Int. Cl. A61M 25/00

(52) U.S. Cl. CPC A61M 25/0043
USPC 604/508; 604/284

(57) ABSTRACT

Disclosed herein are various embodiments of catheters configured to reduce, minimize or prevent occlusion of blood flow resulting from thrombus or fibrin sheath formation, related systems, and methods of use.

### TECHNICAL FIELD

The present disclosure relates generally to medical devices. In particular, the present disclosure relates to catheters with anti-occlusion features suitable for use in vascular access procedures. More particularly, the catheters may be configured to reduce, minimize, or prevent the occlusion of blood flow resulting from thrombus or fibrin sheath formation.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 illustrates a plan view of an exemplary catheter disclosed herein.

FIG. 2 is a plan view of a distal portion of the catheter of FIG. 1 enlarged relative to FIG. 1.

FIG. 3A is a plan view of a distal portion of one embodiment of a catheter, wherein the distal ports of the first lumen part and the second lumen part are perpendicularly oriented relative to the longitudinal axis of the catheter, and the length of the first lumen part extends distally relative to the second lumen part.

FIG. 3B is a plan view of a distal portion of one embodiment of a catheter, wherein the distal ports of the first lumen part and the second lumen part are obliquely oriented relative to the longitudinal axis of the catheter. The acutely angled side of the first lumen part is disposed adjacent to the obtusely angled side of the second lumen part, and the length of the first lumen part extends distally relative to the second lumen part.

FIG. 3C is a plan view of a distal portion of one embodiment of a catheter, wherein the distal ports of the first lumen part and the second lumen part are obliquely oriented relative to the longitudinal axis of the catheter. The obtusely angled side of the first lumen part is disposed adjacent to the acutely angled side of the second lumen part, and the length of the first lumen part extends distally relative to the second lumen part.

FIG. 3D is a plan view of a distal portion of one embodiment of a catheter, wherein the distal ports of the first lumen part and the second lumen part are obliquely oriented relative to the longitudinal axis of the catheter. The obtusely angled side of the first lumen part is disposed adjacent to the obtusely angled side of the second lumen part, and the length of the first lumen part extends distally relative to the second lumen part.

FIG. 3E is a plan view of a distal portion of one embodiment of a catheter, wherein the distal ports of the first lumen part and the second lumen part are perpendicularly oriented relative to the longitudinal axis of the catheter, and the length of the first lumen part is substantially similar to the length of the second lumen part.

FIG. 3F is a plan view of a distal portion of one embodiment of a catheter, wherein the distal ports of the first lumen part and the second lumen part are obliquely oriented relative to the longitudinal axis of the catheter. The acutely angled side of the first lumen part is disposed adjacent to the obtusely angled side of the second lumen part, and the length of the second lumen part extends distally relative to the first lumen part.

FIG. 4A is a cross-sectional view along plane 4A-4A of FIG. 2, illustrating one embodiment of the flow passages through the lumen parts and the catheter walls.

FIG. 4B is a cross-sectional view along plane 4B-4B of FIG. 2, illustrating another embodiment of the flow passages through the lumen parts and the catheter walls.

FIG. 5A is a top plan view of a distal portion of one embodiment of a catheter, wherein the distal tips of the first lumen part and the second lumen part are tapered.

FIG. 5B is a side view of the distal portion of FIG. 5A.

FIG. 5C is a bottom plan view of the distal portion of FIG. 5A.

FIG. 6 is a perspective view of a distal portion of one embodiment of a catheter, wherein the distal tips of the first lumen part and the second lumen part are symmetrical with biased-cut distal ports.

### DETAILED DESCRIPTION

For the purposes of promoting an understanding of the principles of the disclosure provided herein, reference will now be made to the embodiment illustrated in the drawings and specific language will be used to describe the same. It will be readily understood with the aid of the present disclosure that the components of the embodiments, as generally described and illustrated in the figures herein, could be arranged and designed in a variety of configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Devices, systems, and methods for use of a self-centering catheter with anti-occlusion features are described herein. The methods, systems, and devices disclosed are suited for use in connection with any medical device, equipment or machinery configured or used to provide a subject with a vascular-access procedure. For example, the catheter disclosed herein may be used in conjunction with any commercially available hemodialysis machine or equipment to provide an effective dialysis procedure to a subject.

The present disclosure provides a catheter with a non-linear configuration at least along a distal portion thereof that is implanted in a vascular structure of a subject. In some embodiments, the catheter discussed herein may be coupled to a dialysis machine to provide patients with end stage renal disease (ESRD) with hemodialysis treatments. Applications of the catheter may include long-term vascular access procedures, including, but not limited to, hemodialysis and apheresis. Applications in short-term vascular access procedures are also contemplated.

For use in vascular access procedures, the catheter is inserted percutaneously into the vascular system. To provide a patient with an effective dialysis treatment, a sufficient volume of blood over a period of time must be removed from the patient, effectively cleansed or purified, and returned to the patient. In addition, to facilitate an effective dialysis procedure, the catheters disclosed herein are configured to have two lumen parts, such that the contaminated blood and the cleansed blood are kept substantially separate. The first and second lumen parts provide flow out of and into the patient's vascular system. In one embodiment, the first lumen part aspirates blood from a blood vessel of a patient to a dialysis machine where it is processed to remove toxins or waste, and the second lumen part returns the cleansed or purified blood to the patient. In an alternative embodiment, the second lumen part aspirates blood from a blood vessel of the patient to a dialysis machine where it is cleansed to remove toxins or waste, and the first lumen part infuses the cleansed or purified blood into the patient.

As used herein, flow into the body is also referred to as “venous” flow, and flow out of the body is called “arterial” flow. In one embodiment, a lumen part that aspirates blood from a blood vessel of a patient may be referred to as an arterial lumen, and a lumen part that infuses blood into the patient may be referred to as a venous lumen.

A common problem associated with dual lumen catheters used for hemodialysis is the partial or complete occlusion of the distal tips due to thrombus and fibrin sheath formation. Thrombus and fibrin sheath formation around the distal tips of the catheter can partially or completely occlude the flow passages of the catheter, which may result in decreased flow rate and lowered efficiency of a dialysis procedure, or a substantial or complete loss in catheter function. The configurations of the catheters disclosed herein provide anti-occlusion features that reduce, minimize, or prevent the formation of thrombus or fibrin sheath around the distal tips of the catheter.

The non-linear distal portion of the catheter includes first and second lumen parts that each include at least one flow passage to permit fluid flow therethrough. The non-linear configuration of the distal portion improves delivery and return of the fluid from the vascular structure and assists to prevent the formation of thrombus or fibrin sheath that could impede catheter performance around the distal tips of the catheter. In some embodiments, the non-linear configuration of the distal portion is a substantially circular or double D-shaped configuration. In such an embodiment, the outer wall surface of the catheter or of the lumen parts limits continuous contact between the catheter and the vessel wall to two points on the catheter walls, and suspends the distal tips of the catheter in the flowing blood. In some embodiments, contact between the vessel wall and the distal portion of the catheter is substantially limited to an apex of a concave bend with respect to the longitudinal axis of the first lumen, and an apex of a concave bend with respect to the longitudinal axis of the second lumen. This configuration allows the catheter to be inserted into a blood vessel of a patient, such that the substantially double D-shaped or circular configuration of the distal portion centers the distal tips of the catheter within the vessel lumen and away from the vessel wall, which minimizes or prevents thrombus or fibrin sheath formation around the distal tips of the catheter.

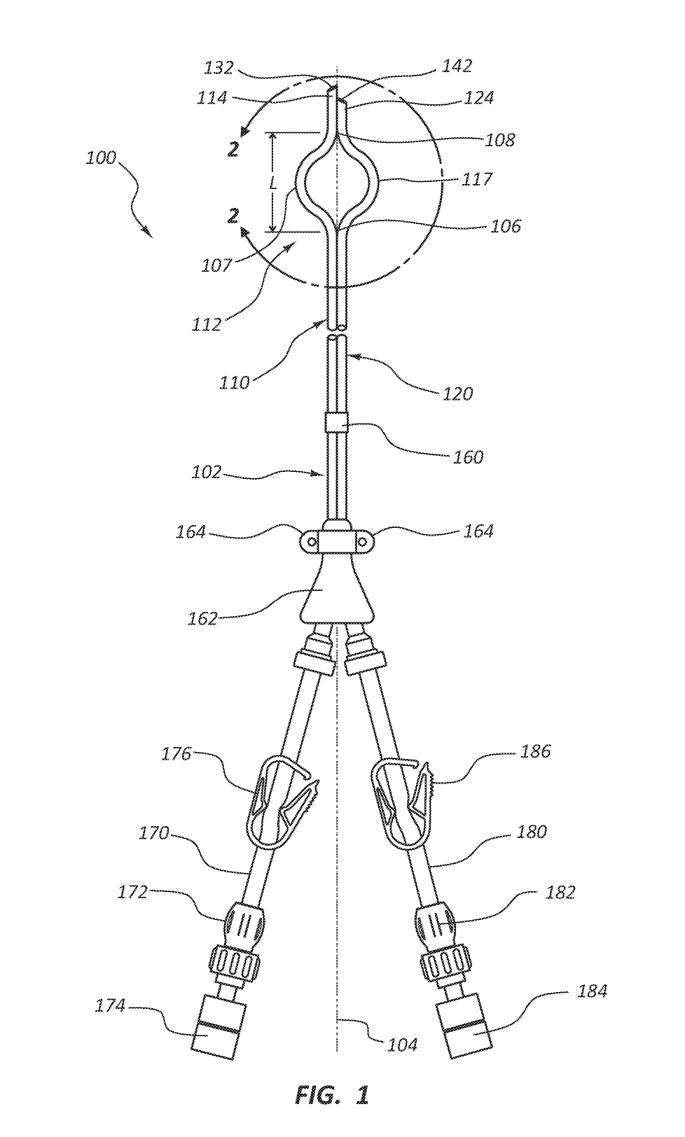

Shown in FIG. 1 is a catheter 100 having an elongated body that includes lumen parts 110, 120 that can be coupled together or disposed alongside one another along a proximal portion 102. The proximal portion 102 can extend distally and proximally along a longitudinal axis 104. The proximal portion 102 may include a cuff 160 extending around lumen parts 110, 120. The cuff 160 provides a scaffold for tissue ingrowth at the location where the catheter 100 enters the skin of the patient. The cuff 160 may be fabricated from any suitable medical-grade material including, but not limited to, polyester felt and the like. In certain embodiments, the cuff 160 may be omitted from the catheter 100. The proximal portion 102 may also include a hub 162 that connects to the proximal ends of the respective lumen parts 110, 120. The hub 162 includes a wing 164 that defines openings for receiving sutures to allow securement of the catheter 100 to the patient after implantation. The hub 162 may also be configured to maintain the separation of flow passages 132, 142 of lumen parts 110, 120. The flow passages 132, 142 are in fluid communication with respective first and second leads 170, 180 extending proximally from the hub 162. In an embodiment, the leads 170, 180 are made from flexible tubing, and may include female fittings 172, 182, respectively at their proximal ends. The male fittings 174, 184 are removably engageable to the respective female fittings 172, 182 to provide a cap to maintain the integrity and sterility of the female fittings 172, 182. The leads 170, 180 may also include clamps 176, 186, respectively, that releasably clamp the respective leads 170, 180 to restrict or allow fluid flow through the flow passages 132, 142 of the lumen parts 110, 120. It will be appreciated by one of ordinary skill in the art having the benefit of this disclosure that the proximal portion 102 of the catheter 100 may be modified.

As illustrated herein, the lumen parts 110, 120 of the catheter 100 may be configured to center the distal tips 114, 124 in the vessel, such as the superior vena cava (SVC), and away from the vessel walls of the patient to reduce, minimize, or prevent thrombus or fibrin sheath formation around the distal tips 114, 124 of the catheter 100. In some embodiments, the catheter 100 can be configured such that even if the distal portion 112 of the catheter 100 were to be inserted through the lumen of the SVC and placed in the right atrium, the substantially non-linear configuration of the lumen parts 110, 120 along length L and the parallel, substantially linear profile of the distal tips 114, 124 extending along the longitudinal axis 104 from the convergence 108 are likely to reduce, minimize, or prevent the occlusion of the flow passages 132, 142 of the distal tips 114, 124.

The catheter 100 can be fabricated from any suitable bio-compatible material, including, but not limited to, silicone, polyurethane, polyurethane-polycarbonate copolymer, or any other plastic or polymer material. The catheter 100 may also include an anti-microbial or anti-infection agent coating, such as silver, chlorhexidine, rifampin, minocycline, methylene blue, and the like.

The catheter 100 can be of any suitable size for placement in a vessel structure. It will be appreciated by those of skill in the art having the benefit of this disclosure that the size of the catheter 100 may be configured to fit within a vessel. In certain embodiments relating to dialysis, the catheter size may be between about 13 to about 16 French circumference. In other embodiments relating to infusing fluid and drawing blood samples from the central veins, the catheter size may be between about 5 to about 12 French circumference. Other catheter sizes are also contemplated.

The configuration of the catheter 100 may be manipulated to facilitate placement of the catheter 100 in a subject. In some embodiments, the double D-shaped or circular configuration of the catheter 100 is compressible, such that catheter 100 may be compressed into a substantially linear profile. In one embodiment, the catheter 100 may be compressed into a substantially linear profile using a sheath. The catheter 100 may be inserted into a sheath and threaded through the patient's vasculature for placement in the desired blood vessel, such as the SVC. In an alternative embodiment, the catheter 100 may be placed over a single guidewire or dual guidewires, with or without stylets, to facilitate placement of the catheter 100 in a subject.

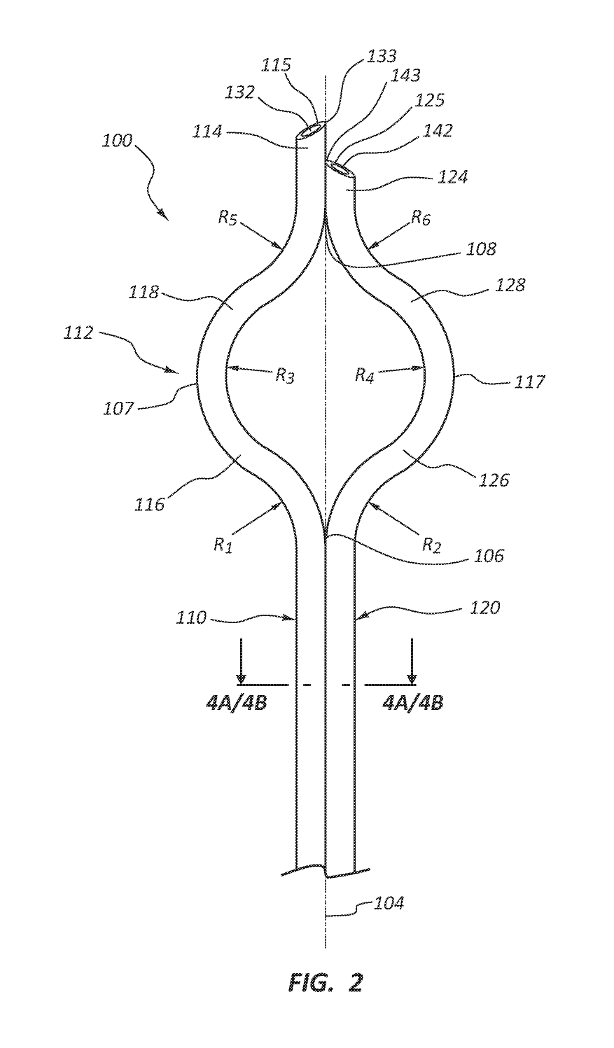

FIG. 2 is a plan view of a distal portion of the catheter of FIG. 1 enlarged relative to FIG. 1. As also shown in FIG. 2, at the distal portion 112 of the catheter 100, lumen parts 110, 120 form a bifurcation 106 that forms a general Y-shape that separates the two lumen parts 110, 120. The separated lumen parts 110, 120 extend for a length L and meet at a convergence 108 to form a general inverted Y-shape. The bifurcation 106 and convergence 108 of the lumen parts 110, 120 form a general non-linear profile along the length L. In some embodiments, the bifurcation 106 and convergence 108 of the lumen parts 110, 120 form a substantially circular or ellipsoid configuration along the length L. In certain embodiments, the diameter of the non-linear portion of the catheter 100 is between about the diameter of the vein into which it is placed when the vein is not distended and about the diameter of the vein when it is fully distended. In one embodiment, the diameter of the non-linear portion of the catheter 100 can range from between about 2 to about 3 centimeters for catheters 100 placed within the superior or inferior vena cava. As will be appreciated by one of skill in the art, the diameter of the non-linear portion of the catheter 100 may be smaller for catheters 100 placed in more peripheral veins. When the lumen parts 110, 120 contact at the convergence 108, the lumen parts 110, 120 extend distally from the convergence 108 in a substantially linear profile relative to the longitudinal axis 104. In the illustrated embodiment, the lumen parts 110, 120 form a substantially double D-shaped or circular configuration to make it less likely that the distal tips 114, 124 can be encased in or occluded by a thrombus or fibrin sheath formed by the patient's body.

For example, the first lumen part 110 forms a first portion 116 that extends from bifurcation 106 along a bend defined by radius R1 to a first apex 107. The second lumen part 120 forms a first portion 126 that extends from bifurcation 106 along a bend that is defined by radius R2 to a second apex 117. Radii R1 and R2 are located on the side of the respective lumen parts 110, 120 away from the longitudinal axis 104 so that the lumen parts 110, 120 separate and diverge distally away from the longitudinal axis 104 and form a convex relationship toward the longitudinal axis 104. Radius R1 and radius R2 can be the same, or can differ from one another.

The first lumen part 110 also includes a second portion 118 forming a reverse curve-like shape relative to the first portion 116 from the first apex 107. The second portion 118 extends from the first apex 107 along a bend defined by a radius R3 to the convergence 108. The second lumen part 120 also includes a second portion 128 that forms a reverse curve-like shape relative to the first portion 126. The second portion 128 extends from the second apex 117 along a bend defined by a radius R4 to the convergence 108. Radii R3 and R4 are located toward the longitudinal axis 104 and form a concave relationship oriented toward the longitudinal axis 104. Radius R3 and radius R4 can be the same, or can differ from one another.

The lumen parts 110, 120 contact at the convergence 108 and extend distally in a parallel, substantially linear profile along the longitudinal axis 104 to form the distal tips 114, 124. In some embodiments, the convergence 108 of the lumen parts 110, 120 may be formed by radii R5 and R6. Radii R5 and R6 are located on the side of the respective lumen parts 110, 120 away from the longitudinal axis 104 so that the lumen parts 110, 120 converge distally and form a convex relationship toward the longitudinal axis 104. Radius R5 and radius R6 can be the same, or can differ from one another. The lumen parts 110, 120 contact at the convergence 108 and extend distally in a parallel, substantially linear profile to form the distal tips 114, 124 of the catheter 100.

The triple-bend configuration of distal portion 112 provides a non-linear profile that assists in preventing or inhibiting the formation of a thrombus or fibrin sheath that encases the distal portion 112 and/or the distal tips 114, 124 when placed in a the body of a subject. The diverging and converging relationship between the lumen parts 110, 120 provides a non-linear profile for the lumen parts 110, 120 along the distal portion 112. It can be appreciated by those of skill in the art having the benefit of this disclosure that the bends are curved along a radius or radii to provide a smooth transition between the first portions 116, 126 and the second portions 118, 128 of the catheter 100 to prevent or reduce the formation of sharp bends or kinks in the lumen parts 110, 120 that could restrict or prevent flow through the flow passages 132, 142 thereof. In some embodiments, the divergence and convergence of the first lumen 110 and second lumen 120 form concave bends along the lumen parts 110, 120 with respect to the longitudinal axis 104 of the catheter 100.

The radii R1, R2, R3, R4, R5, and R6 are provided as examples of the curvature of the bends of the lumen parts 110, 120. The radii R1, R2, R3, R4, R5, and R6 may be any measurement capable of maintaining a suitable flow rate for hemodialysis through the flow passages 132, 142 of the lumen parts 110, 120. In one embodiment, the radii R1, R2, R3, R4, R5, and R6 are the same. In another embodiment, the radii R1, R2, R3, R4, R5, and R6 differ from one another. In an alternative embodiment, a combination of radii R1, R2, R3, R4, R5, and R6 have the same measurement, while the remaining radii differ in measurement. In particular embodiments, the radii R1, R2, R3, R4, R5, and R6 are configured to achieve or maintain a flow rate of about 300 mL/min. In one embodiment, radii R1, R2, R3, R4, R5, and R6 can each independently range from between about 0.5 to about 3.0 centimeters. In another embodiment, radii R1, R2, R3, R4, R5, and R6 can each independently range from between about 0.5 to about 1.5 centimeters.

The lengths of the distal tips 114, 124 are parallel to each other and extend along the longitudinal axis 104 of the catheter 100. In some embodiments, the first distal port 115 of the first distal tip 114 and the second distal port 125 of the second distal tip 124 are obliquely oriented relative to the longitudinal axis 104 of the catheter 100. The acutely angled side 133 of the first distal tip 114 is disposed adjacent to the acutely angled side 143 of the second distal tip 124, while the first distal tip 114 projects distally from the second distal tip 124 along the longitudinal axis 104 of the catheter 100. In certain embodiments, the distance of separation between the first distal tip 114 and the second distal tip 124 along the longitudinal axis 104 is about 0.5 inches. In some embodiments, the longitudinal separation between first 114 and second 124 distal tips ranges between zero to 1.0 inches, such as from 0.25 to 0.75 inches. Other longitudinal separation between the first 114 and second 124 distal tips are also contemplated. In one embodiment, the kinetic energy of the blood flowing out of the venous lumen delivers it away from the inflow of the arterial lumen, such that there is no demonstrable recirculation. In yet another embodiment, the catheter 100 is configured to have a longitudinal separation that results in minimal or no demonstrable recirculation at about 200 to about 400 mL/min flow in the middle of the fluid simulating the flow of blood in the middle of the vena cava. In some embodiments, no demonstrable recirculation can be, for example, less than about 1% recirculation, less than about 3% recirculation, and less than about 5% recirculation. In particular embodiments, the distance of separation between the distal tips 114, 124 along the longitudinal axis 104 may be configured to achieve or maintain a flow rate of at least about 300 mL/min through the catheter 100.

In an embodiment of a method or system for providing hemodialysis procedure to a patient, a dialysis machine (not shown) may be coupled to the catheter 100, and the catheter 100 may be coupled to a patient. The flow passages 132, 142 of the catheter 100 maintain fluid communication between the dialysis machine and the patient. In one embodiment, the first lumen part 110 aspirates blood from a blood vessel of the patient to a dialysis machine where it is processed to remove toxins or waste, and the second lumen part 120 returns the cleansed or purified blood to the patient. In another embodiment, the second lumen part 120 aspirates blood from a blood vessel of the patient to a dialysis machine where it is cleansed to remove toxins or waste, and the first lumen part 110 returns the cleansed or purified blood to the patient.

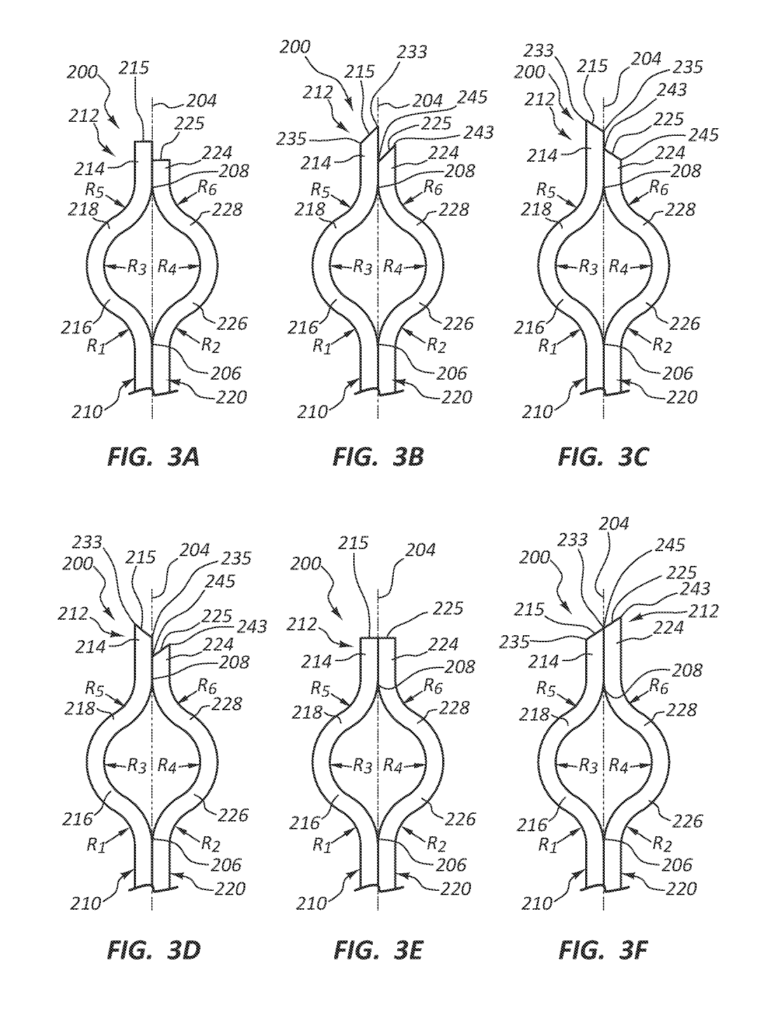

FIGS. 3A-3F provide plan views of various embodiments of distal portions 112 of exemplary catheters 100, as disclosed herein.

As shown in FIGS. 3A-3F, the first lumen part 210 forms a first portion 216 that extends from bifurcation 206 along a bend defined by radius R1. The second lumen part 220 also forms a first portion 226 that extends from bifurcation 206 along a bend that is defined by radius R2. Radii R1 and R2 are located on the side of the respective lumen parts 210, 220 away from the longitudinal axis 204 so that the lumen parts 210, 220 separate and diverge distally and radially away from the longitudinal axis 204 and form a convex relationship toward the longitudinal axis 204. Radius R1 and radius R2 can be the same, or can differ from one another.

The first lumen part 210 also includes a second portion 218 that forms a reverse curve-like shape relative to the first portion 216. The second portion 218 of the first lumen part 210 extends along a bend defined by a radius R3 to the convergence 208. The second lumen part 220 also includes a second portion 228 that forms a reverse curve-like shape relative to the first portion 226. The second portion 228 of the second lumen part 220 extends along a bend defined by a radius R4 to the convergence 208. Radii R3 and R4 are located toward the longitudinal axis 204 and form a concave relationship toward the longitudinal axis 204. Radius R3 and radius R4 can be the same, or can differ from one another.

The lumen parts 210, 220 are configured to contact at the convergence 208 and extend distally in a parallel, substantially linear profile along the longitudinal axis 204 to form the distal tips 214, 224. The convergence 208 of the lumen parts 210, 220 may be formed by radii R5 and R6. Radii R5 and R6 are located on the side of the respective lumen parts 210, 220 away from the longitudinal axis 204 such that the lumen parts 210, 220 converge distally and form a convex relationship toward the longitudinal axis 204. Radius R5 and radius R6 can be the same, or can differ from one another. The lumen parts 210, 220 contact at the convergence 208 and extend distally in a parallel, substantially linear profile to form the distal tips 214, 224 of catheter 200.

It will be appreciated by those of skill in the art having the benefit of this disclosure that the orientation of the distal ports 215, 225 can be modified. In certain embodiments, the distal ports 215, 225 of the catheter 200 are cut at the same angle. It will also be appreciated by those of skill in the art that the distance of separation between the distal tips 214, 224, along the longitudinal axis 204, can be modified. As is similar to FIG. 1, in certain embodiments, the distance of separation between the distal tips 214, 224, along the longitudinal axis 204, is about 0.5 inches. In some embodiments, the longitudinal separation between first 214 and second 224 distal tips ranges between zero to 1.0 inches, such as from 0.25 to 0.75 inches. Other longitudinal separations between the first 214 and second 224 distal tips are also contemplated. In one embodiment, the kinetic energy of the blood flowing out of the venous lumen delivers it away from the inflow of the arterial lumen, such that there is no demonstrable recirculation. In yet another embodiment, the catheter 200 is configured to have a longitudinal separation that results in minimal or no demonstrable recirculation at about 200 to about 400 mL/min flow in the middle of the fluid simulating the flow of blood in the middle of the vena cava. In some embodiments, no demonstrable recirculation can be, for example, less than about 1% recirculation, less than about 3% recirculation, and less than about 5% recirculation. In particular embodiments, the distance of separation between the distal tips 214, 224 along the longitudinal axis 204 may be configured to achieve or maintain a flow rate of at least about 300 mL/min through the catheter 200.

Shown in FIG. 3A is an embodiment of a distal end portion 212 where the first distal tip 214 and the second distal tip 224 extend distally along the longitudinal axis 204 from the convergence 208. The lengths of the distal tips 214, 224 are parallel to each other and extend along the longitudinal axis 204 of the catheter 200. The first distal port 215 and the second distal port 225 are both perpendicularly oriented relative to the longitudinal axis 204, and the plane of the first distal port 215 is substantially parallel to the plane of the second distal port 225. The first distal tip 214 is disposed adjacent to the second distal tip 224, and projects a distance along the longitudinal axis 204 of the catheter 200 from the second distal port 225.

FIG. 3B illustrates an embodiment of a distal end portion 212 where the first distal tip 214 and the second distal tip 224 extend distally along the longitudinal axis 204 from the convergence 208. The lengths of the distal tips 214, 224 are parallel to each other and extend along the longitudinal axis 204 of the catheter 200. The first distal port 215 and the second distal port 225 are both obliquely oriented relative to the longitudinal axis 204, and the plane of the first distal port 215 is substantially parallel to the plane of the second distal port 225. The acutely angled side 233 of the first lumen part 210 and the obtusely angled side 245 of the second lumen part 220 are positioned adjacent to the longitudinal axis 204, while the obtusely angled side 235 of the first lumen part 210 and the acutely angled side 243 of the second lumen part 220 are spaced apart from the longitudinal axis 204. The acutely angled side 233 of the first lumen part 210 is disposed substantially adjacent to the obtusely angled side 245 of the second lumen part 220. The first distal tip 214 is disposed adjacent to the second distal tip 224, and projects a distance along the longitudinal axis 204 of the catheter 200 from the second distal port 225.

Shown in FIG. 3C is another embodiment of a distal end portion 212 where the first distal tip 214 and the second distal tip 224 extend distally along the longitudinal axis 204 from the convergence 208. The lengths of the distal tips 214, 224 are parallel to each other and extend along the longitudinal axis 204 of the catheter 200. The first distal port 215 and the second distal port 225 are both obliquely oriented relative to the longitudinal axis 204, and the plane of the first distal port 215 is substantially parallel to the plane of the second distal port 225. The obtusely angled side 235 of the first lumen part 210 and the acutely angled side 243 of the second lumen part 220 are positioned adjacent to the longitudinal axis 204, while the acutely angled side 233 of the first lumen part 210 and the obtusely angled side 245 of the second lumen part 220 are spaced apart from the longitudinal axis 204. The obtusely angled side 235 of the first lumen part 210 is disposed substantially adjacent to the acutely angled side 243 of the second lumen part 220. The first distal tip 214 is disposed adjacent to the second distal tip 224, and projects a distance along the longitudinal axis 204 of the catheter 200 from the second distal port 225.

FIG. 3D illustrates yet another embodiment of the distal end portion 212 where the first distal tip 214 and the second distal tip 224 extend distally along the longitudinal axis 204 from the convergence 208. The lengths of the distal tips 214, 224 are parallel to each other and extend along the longitudinal axis 204 of the catheter 200. The first distal port 215 and the second distal port 225 are both obliquely oriented relative to the longitudinal axis 204, such that the plane of the first distal port 215 transverses the plane of the second distal port 225. The obtusely angled 235 side of the first lumen part 210 and the obtusely angled side 245 of the second lumen part 220 are positioned adjacent to the longitudinal axis 204, while the acutely angled side 233 of the first lumen part 210 and the acutely angled side 243 of the second lumen part 220 are spaced apart from the longitudinal axis 204. The obtusely angled side 235 of the first lumen part 210 is disposed substantially adjacent to the obtusely angled side 245 of the second lumen part 220. The first distal tip 214 is disposed adjacent to the second distal tip 224, and projects a distance along the longitudinal axis 204 of the catheter 200 from the second distal port 225.

Shown in FIG. 3E is another embodiment of the distal end portion 212 where the first distal tip 214 and the second distal tip 224 extend distally along the longitudinal axis 204 from the convergence 208. The lengths of the distal tips 214, 224 are parallel to each other and extend along the longitudinal axis 204 of the catheter 200. The first distal port 215 and the second distal port 225 are both perpendicularly oriented relative to the longitudinal axis 204, such that both distal ports 215, 225 are in the same plane. The first distal tip 214 is disposed substantially adjacent to the second distal tip 224, and the first distal tip 214 is the same length as the second distal tip 224. The first distal tip 214 is disposed adjacent to the second distal tip 224, and projects a distance along the longitudinal axis 204 of the catheter 200 from the second distal port 225.

FIG. 3F illustrates another embodiment of the distal end portion 212 where the first distal tip 214 and the second distal tip 224 extend distally along the longitudinal axis 204 from the convergence 208. The lengths of the distal tips 214, 224 are parallel to each other and extend along the longitudinal axis 204 of the catheter 200. The first distal port 215 and the second distal port 225 are both obliquely oriented relative to the longitudinal axis 204, such that both distal ports 215, 225 are in the same plane. The acutely angled side 233 of the first lumen part 210 and the obtusely angled side 245 of the second lumen part 220 are positioned adjacent to the longitudinal axis 204, while the obtusely angled side 235 of the first lumen part 210 and the acutely angled side 243 of the second lumen part 220 are spaced apart from the longitudinal axis 204. The acutely angled side 233 of the first lumen part 210 is disposed substantially adjacent to the obtusely angled side 245 of the second lumen part 220. The second distal tip 224 is disposed adjacent to the first distal tip 214, and projects a distance along the longitudinal axis 204 of the catheter 200 from the first distal port 215.

The distal tips 214, 224 extending from the convergence 208 may be coupled or secured to one another with an adhesive, an extrusion technique, fusion, mechanical fasteners, and the like. In some embodiments, the distal tips 214, 224 are coupled or secured using a bond material with low adhesion strength. In certain embodiments, the bond between distal tips 214, 224 is weaker than a bond between proximal portions of the first 210 and second 220 lumen parts. In other embodiments, the distal tips are disposed adjacent to each other without being coupled or secured to one another. In yet another embodiment, the distal tips are held in place using wires or stiffening mechanisms inserted through the length of the catheter walls.

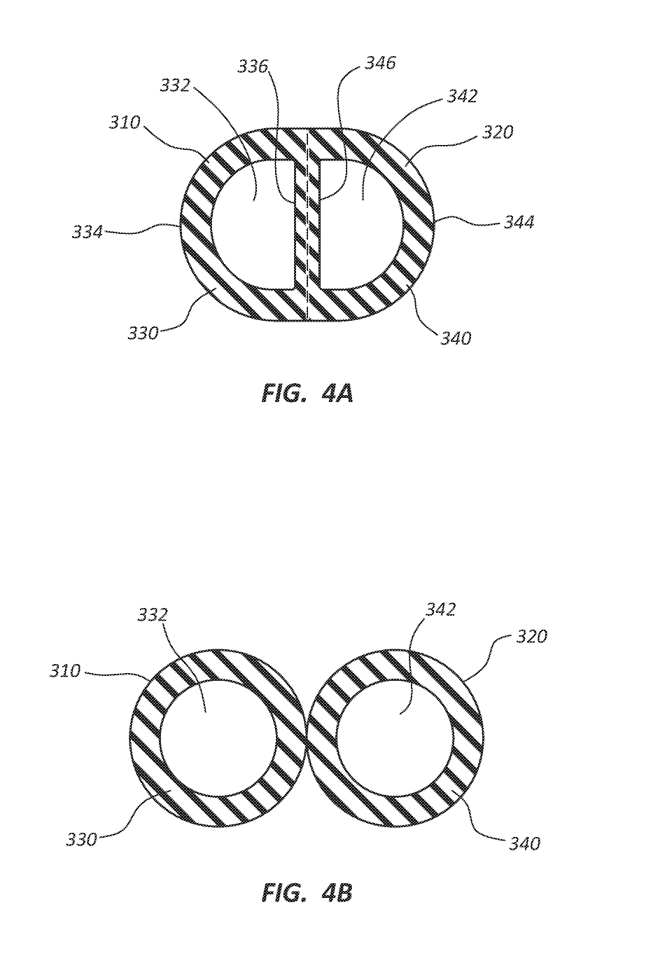

FIG. 4A provides a cross-sectional view along line 4A-4A of FIG. 2 that illustrates an embodiment of the flow passages 332, 342 through the lumen parts 310, 320 and the catheter walls 330, 340. The first lumen part 310 includes a D-shaped wall 330 that defines a flow passage 332 extending therethrough. The wall 330 includes an arcuate portion 334 and a linear portion 336 extending between opposite sides of the arcuate portion 334. Similarly, the second lumen part 320 includes a D-shaped wall 340 that defines a flow passage 342 extending therethrough. The D-shaped wall 340 includes an arcuate portion 344 and a linear portion 346 extending between opposite sides of the arcuate portion 344. The lumen parts 310, 320 are positioned with the linear portions adjacent to each other along the longitudinal axis, at least proximally of bifurcation and distally of convergence.

FIG. 4B illustrates a cross-sectional view along line 4B-4B of FIG. 2 that illustrates another embodiment of the flow passages 332, 342 through the lumen parts 310, 320 and the catheter walls 330, 340. The first lumen part 310 includes a substantially circular wall 330 that defines a flow passage 332 extending therethrough. Similarly, the second lumen part 320 includes a substantially circular wall 330 that defines a flow passage 332 extending therethrough. The lumen parts 310, 320 are positioned such that the length of the lumen parts 310, 320 are positioned adjacent to each other along the longitudinal axis, at least proximally of bifurcation and distally of convergence. It will be appreciated that the flow passages 332, 342 may also be configured to have different shapes from the flow passages 332, 342 illustrated in FIGS. 4A and 4B.

The lumen parts 310, 320 proximal of bifurcation can be coupled or secured to one another using an adhesive, an extrusion technique, fusion, mechanical fastener, and the like. In some embodiments, the lumen parts 310, 320 proximal of bifurcation can be heat-welded together at approximately 230° F.

The lumen parts 310, 320 distal of convergence may also be coupled to one another using an adhesive, extrusion technique, fusion, mechanical fasteners, and the like. In some embodiments, the lumen parts 310, 320 distal of convergence may be coupled using a bonding material with low adhesion strength, such that the adhesion strength is less than that of the lumen parts 310, 320 proximal of the bifurcation. In an alternative embodiment, the lumen parts 310, 320 distal of convergence may be disposed in a side-by-side relation along the longitudinal axis of the catheter without coupling the lumen parts 310, 320 to one another.

In yet another embodiment, the lumen parts 310, 320 distal of convergence may be held in position using wires or stiffening mechanisms (not shown) inserted through the length of the catheter walls 330, 340. The wires or stiffening mechanisms may be of varying sizes and lengths suitable for use in this context. In one embodiment, the wires or stiffening mechanisms may be incorporated into the catheter walls 330, 340 along the distal portion of the catheter 300. In some embodiments, the wires or stiffening mechanisms are incorporated into the catheter walls 330, 340 along the double D-shaped or circular configuration of the distal portion. The double D-shaped or circular configuration of the distal portion including the wires or stiffening mechanisms may be flexible. In certain embodiments, the double D-shaped or circular configuration of the distal portion may be compressed into a substantially linear profile, which may aid with placing the catheter in a subject.

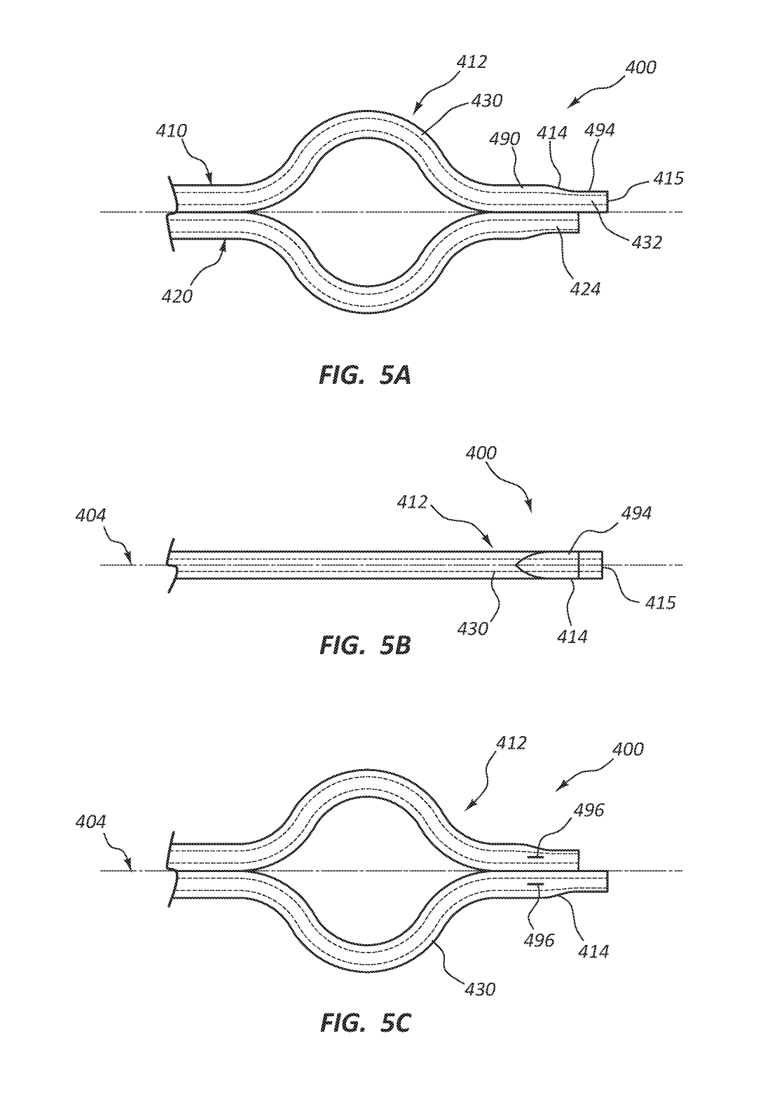

Various arrangements for the distal tips are contemplated. In one embodiment, the configurations of the respective lumen parts 310, 320 shown in FIGS. 4A and 4B are carried along the lumen parts 310, 320 through the distal ports. In another embodiment, shown in FIGS. 5A-5C, the distal tips 414, 424 include a distally tapered configuration.

FIG. 5A provides a top plan view of a distal portion 412 of one embodiment of a catheter 400, wherein the distal tips 414, 424 of the lumen parts 410, 420 are tapered. As illustrated in FIG. 5A, the catheter wall 430 includes a first thickness along a proximal part 490 of the distal tip 414. The thickness of the catheter wall 430 tapers along a distal part 494 of the distal tip 414 from the end of the first thickness toward the distal port 415, where the flow passage 432 remains constant through the distal tip 414 to maximize the area available for flow. In some embodiments, the reduced wall thickness along the distal part 494 of the distal tip 414 increases flexibility of the distal tip 414 and provides further protection to the vascular structure from trauma should the distal tip 414 contact the vessel wall or other anatomic structure.

Shown in FIG. 5B is a side elevation view of the distal tip 414 of the catheter 400 of FIG. 5A. The curvature along the length of the distal portion 412 lies in the same plane as the longitudinal axis 404 of the catheter 400. The thickness of the catheter wall 430 tapers along the distal part 494 of the distal tip 414 from the end of the first thickness toward the distal port 415.

FIG. 5C illustrates a bottom plan view of the distal portion 412 of the catheter 400 of FIG. 5A. In some embodiments, the catheter 400 may include a side port 496 extending along the catheter wall 430 parallel to the longitudinal axis 404 of the catheter 400. The side port 496 forms an elongated slit that is normally closed, but may be opened upon application of sufficient fluid pressure to allow at least some fluid flow therethrough. In another embodiment, the side port 496 is omitted from the distal tip 414. In other embodiments, multiple side ports 496 are provided in the distal tip 414.

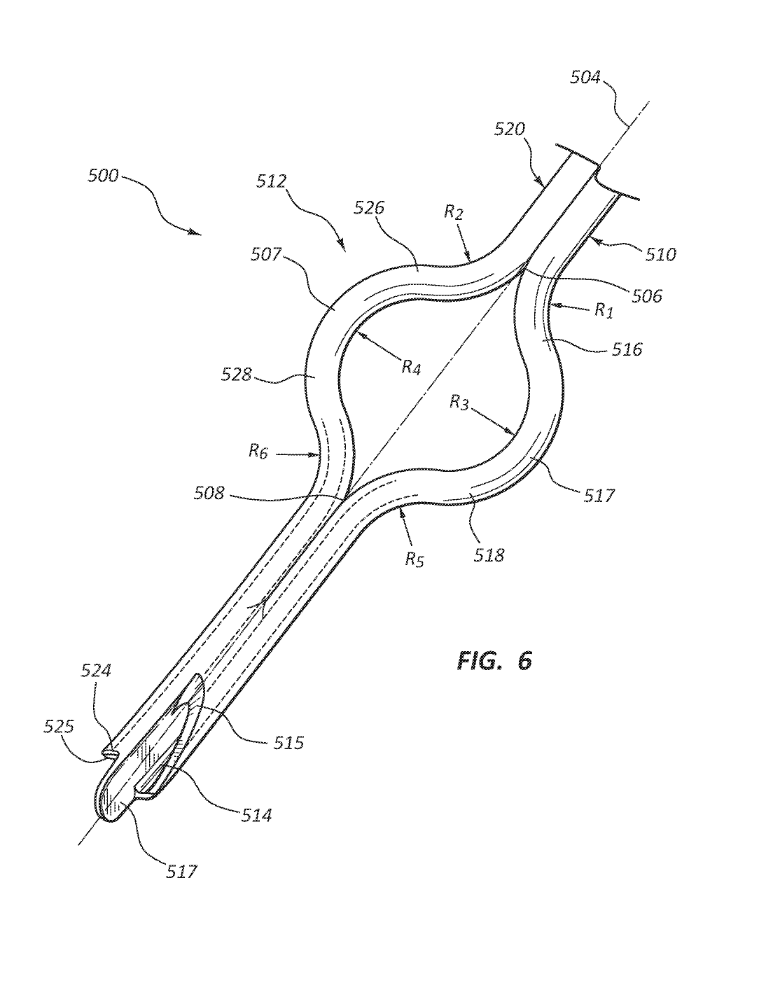

FIG. 6 illustrates a perspective view of another embodiment of a distal portion 512 of a catheter 500. As illustrated in FIG. 6, the first lumen part 510 forms a first portion 516 that extends from bifurcation 506 to a first apex 507 along a bend defined by radius R1. The second lumen part 520 also forms a first portion 526 that extends from bifurcation 506 to a second apex 517 along a bend that is defined by radius R2. Radii R1 and R2 are located on the side of the respective lumen parts 510, 520 away from the longitudinal axis 504 so that the lumen parts 510, 520 separate and diverge distally away from the longitudinal axis 504 and form a convex relationship toward the longitudinal axis 504.

The first lumen part 510 also includes a second portion 518 that forms a reverse curve-like shape relative to the first portion 516. The second portion 518 of the first lumen part 510 extends from the first apex 507 along a bend defined by a radius R3 to the convergence 508. The second lumen part 520 also includes a second portion 528 that forms a reverse curve-like shape relative to the first portion 526. The second portion 528 of the second lumen part 520 extends from the second apex 517 along a bend defined by a radius R4 to the convergence 508. Radii R3 and R4 are located toward the longitudinal axis 504 and form a concave relationship oriented toward the longitudinal axis 504.

The lumen parts 510, 520 are configured to contact at the convergence 508 and extend distally in a parallel, substantially linear profile along the longitudinal axis 504 to form the distal tips 514, 524. The convergence 508 of the lumen parts 510, 520 may be formed by radii R5 and R6. Radii R5 and R6 are located on the side of the respective lumen parts 510, 520 away from the longitudinal axis 504 such that the lumen parts 510, 520 converge distally and form a convex relationship toward the longitudinal axis 504.

As shown in FIG. 6, the distal portion 512 of the catheter 500 may be configured such that the distal tips 514, 524 of the lumen parts 510, 520 are symmetrical with biased-cut distal ports 515, 525. In some embodiments, as illustrated herein in FIG. 6, the plane of the biased-cut first distal port 515 transverses the plane of the biased-cut second distal port 525. In certain embodiments, the angle at which the distal ports 515, 525 are cut can be about 45°. The angle at which the distal ports 515, 525 are cut may also be modified. In particular embodiments, the angles of the cuts can range from about 10° to about 80°, such as from about 30° to about 50°. Other angles are also contemplated. In some embodiments, the angled cuts of the distal ports 515, 525 are configured to facilitate easier insertion of the catheter 500 over a guidewire. As shown in FIG. 6, the distal tips 514, 524 may also be separated by a septum or a median wall 517 that protrudes distally between the distal tips 514, 524. The orientation of the septum or medial wall 517 may be perpendicular to the plane of the bifurcation 506 and convergence 508 of the lumen parts 510, 520. In other embodiments, the median wall 517 may be omitted.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification, are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, inventive aspects lie in a combination of fewer than all features of any single foregoing disclosed embodiment. It will be apparent to those having skill in the art that changes may be made to the details of the above-described embodiments without departing from the underlying principles set forth herein.

1. A catheter, comprising:
a first lumen comprising a proximal portion, a distal portion, and a first distal port; and
a second lumen comprising a proximal portion, a distal portion, and a second distal port;
wherein the proximal portion of the first lumen is disposed adjacent to the proximal portion of the second lumen between a proximal point and a bifurcation point, such that the first lumen and the second lumen diverge at the bifurcation point and converge at a convergence point, and the distal portion of the first lumen is disposed adjacent to the distal portion of the second lumen between the convergence point and a distal point.

2. The catheter of claim 1, wherein the first and second lumens each form a concave bend with respect to the longitudinal axis of the catheter between the bifurcation point and the convergence point.

3. The catheter of claim 2, wherein the first lumen and the second lumen form a substantially circular configuration between the bifurcation point and the convergence point.

4. The catheter of claim 2, wherein the first lumen and the second lumen form a double-D shaped configuration between the bifurcation point and the convergence point.

5. The catheter of claim 1, wherein the distal portion of the first lumen extends along a first portion from the bifurcation point away from the longitudinal axis of the catheter to a first apex, then extends along a second portion from the first apex toward the longitudinal axis of the catheter to the convergence point.

6. The catheter of claim 5, wherein the distal portion of the second lumen extends along a first portion from the bifurcation point away from the longitudinal axis of the catheter to a second apex, then extends along a second portion from the second apex toward the longitudinal axis of the catheter to the convergence point.

7. The catheter of claim 6, wherein the distal portion of the first lumen and the distal portion of the second lumen between the convergence point and the distal point are disposed along the longitudinal axis of the catheter.

8. The catheter of claim 1, wherein the first distal port projects a distance along the longitudinal axis of the catheter from the second distal port.

9. The catheter of claim 1, wherein the distal portion of the first lumen and the distal portion of the second lumen are coupled to each other between the convergence point and the distal point.

10. The catheter of claim 1, wherein the first and second distal ports are obliquely oriented relative to the longitudinal axis.

11. The catheter of claim 9, wherein the first and second distal ports are obliquely oriented relative to the longitudinal axis.

12. The catheter of claim 1, wherein the first and second distal ports are perpendicularly oriented relative to the longitudinal axis.

13. The catheter of claim 9, wherein the first and second distal ports are perpendicularly oriented relative to the longitudinal axis.

14. The catheter of claim 1, wherein the first lumen and the second lumen are symmetrical with biased-cut distal ports, and wherein a median wall protrudes distally between the first lumen and the second lumen along the longitudinal axis of the catheter.

15. A catheter, comprising:
a first lumen comprising a proximal portion, a distal portion, and a first distal port; and
a second lumen comprising a proximal portion, a distal portion, and a second distal port;
wherein the proximal portion of the first lumen is disposed adjacent to the proximal portion of the second lumen between a proximal point and a bifurcation point, and the distal portion of the first lumen is disposed adjacent to the distal portion of the second lumen between a convergence point and a distal point, and the first lumen and the second lumen form a triple-bend configuration between the bifurcation point and the convergence point.

16. The catheter of claim 15, wherein the first distal port projects a distance along the longitudinal axis of the catheter from the second distal port.

17. The catheter of claim 16, wherein the distal portion of the first lumen extends along a first portion from the bifurcation point away from the longitudinal axis of the catheter to a first apex, then extends along a second portion from the first apex toward the longitudinal axis of the catheter to the convergence point, and
wherein the distal portion of the second lumen extends along a first portion from the bifurcation point away from the longitudinal axis of the catheter to a second apex, then extends along a second portion from the second apex toward the longitudinal axis of the catheter to the convergence point.

18. The catheter of claim 16, wherein the first lumen extends from the bifurcation point toward the first apex along a bend defined by a first radius, and the second lumen extends toward the second apex from the bifurcation point along a bend defined by a second radius;
wherein the first lumen extends through the first apex along a bend defined by a third radius, and the second lumen extends through the second apex along a bend defined by a fourth radius; and
wherein the first lumen extends from the convergence point toward the first apex along a bend defined by a fifth radius, and the second lumen extends from the convergence point toward the second apex along a bend defined by a sixth radius.

19. The catheter of claim 15, wherein the first lumen and the second lumen are symmetrical with biased-cut distal ports, and wherein a median wall projects distally between the first lumen and the second lumen along the longitudinal axis of the catheter.

20. The catheter of claim 16, wherein the first and second distal ports are obliquely oriented relative to the longitudinal axis of the catheter.

21. The catheter of claim 16, wherein the first and second distal ports are perpendicularly oriented relative to the longitudinal axis of the catheter.

22. A method of providing a hemodialysis procedure to a subject, the method comprising:
obtaining a catheter comprising a first and second lumen that bifurcate from each other and converge back together at a position adjacent a distal end of the catheter;
inserting the catheter into a vasculature of the subject;
coupling the catheter to a hemodialysis machine; and
actuating the hemodialysis machine.

23. The method of claim 22, wherein obtaining the catheter comprises obtaining a catheter comprising a first apex in the first lumen and a second apex in the second lumen between the bifurcation and convergence of the lumens.

24. The method of claim 23, further comprising:
centering distal ports of the first and second lumen into a body vessel of the vasculature by permitting the first and second apexes to contact a wall of the body vessel.

25. The method of claim 22, further comprising:
aspirating contaminated blood from the vasculature of the subject through the first lumen which is in fluid communication with the hemodialysis machine.

26. The method of claim 25, further comprising:
infusing cleansed blood into the vasculature of the subject through the second lumen which is in fluid communication with the hemodialysis machine.
